(19) United States
(12) Patent Application Publication
(10) Pub. No.: US 2022/0304799 A1
(43) Pub. Date: Sep. 29, 2022

(54) INTRAOCULAR LENS SYSTEM, INTRAOCULAR LENS AND CILIAR BODY IMPLANT

(71) Applicant: Carl Zeiss Meditec AG, Jena (DE)

(72) Inventor: Jan Buchheister, Jena (DE)

(21) Appl. No.: 17/842,091

(22) Filed: Jun. 16, 2022

Related U.S. Application Data
(63) Continuation of application No. PCT/EP2020/087127, filed on Dec. 18, 2020.

(30) Foreign Application Priority Data
Dec. 20, 2019 (DE) 10 2019 135 511.7

(51) Int. Cl. A61F 2/16

(52) U.S. Cl. CPC A61F 2/1648; A61F 2/1629; A61F 2/1635; A61F 2/1694

(57) ABSTRACT

An intraocular lens system for implantation in an eye is provided. The intraocular lens system has a ciliary body implant with a ciliary magnet element, the ciliary body implant being implantable in the eye such that the ciliary magnet element at least partially follows the movements of the ciliary body of the eye. The intraocular lens system also includes an intraocular lens with a lens magnet element. The ciliary body implant and the intraocular lens are formed separately from each other and the intraocular lens system is adapted to control a refractive effect of the intraocular lens via an interaction between the ciliary magnet element and the lens magnet element in the eye. The disclosure also relates to a ciliary body implant and an intraocular lens.

### CROSS REFERENCE TO RELATED APPLICATIONS

This application is a continuation application of international patent application PCT/EP2020/087127, filed Dec. 18, 2020, designating the United States and claiming priority from German patent application DE 10 2019 135 511.7, filed Dec. 20, 2019, and the entire content of both applications is incorporated herein by reference.

### TECHNICAL FIELD

The present disclosure relates to an intraocular lens system, an intraocular lens, and a ciliary body implant. Consequently, the disclosure is in the field of intraocular lenses in particular, more particularly in the field of biomechanically accommodatable intraocular lenses and ophthalmic surgery.

### BACKGROUND

The related art has disclosed intraocular lenses (IOLs) that exhibit an ability to biomechanically accommodate, that is to say the refractive power of the IOL is changed by a mechanical force being exerted by means of muscular tissue and can be adapted to the desired accommodation.

IOLs are frequently implanted into the capsular bag of the eye as this has a low complication rate in comparison with other implantation locations, and the required surgical techniques are well established, with numerous concepts for such biomechanical IOLs being available. For the accommodation, the concepts known from the related art use the naturally triggering force, specifically the change in diameter of the ciliary body or ciliary muscle, only indirectly. Rather, the decisive force transmission is implemented onto the elastic capsular bag via the zonular fibers.

In different eyes or patients, the elasticity of the capsular bag is very different on an individual basis and may change, for example as a result of wound healing processes (e.g., fibrosis) following cataract surgery and as a result of further cell growth (secondary cataract). The treatment of the secondary cataract may also change the capsular bag and, in particular, the elasticity thereof. It is therefore often difficult to find a generally valid, well-functioning biomechanical arrangement which is equally suited to many individual differences in the population and, additionally, to the temporal change of the biological material used for the function.

What makes the matter more difficult is that the properties of the biological material used for the function typically cannot be measured before a cataract operation, which is why it is not possible to adapt to individual conditions.

Moreover, conventional IOL implants typically must span the original capsular bag in order to reduce/avoid fibrosis, necessitating a large implant volume and making small incision dimensions more difficult.

Furthermore, there are concepts for biomechanical accommodatable IOLs, which are implanted outside of the capsular bag into the sulcus or into the vicinity of the sulcus, in direct contact with the ciliary body. In this case, the strength of the ciliary muscle or ciliary body is directly converted into a mechanical movement or hydraulic deformation in order to generate an accommodation of the biomechanical implant. These implants are typically in contact with the iris or carry out relative movements in relation to critical tissue, as a result of which for example pigments can be detached from the iris and these can then for example impede the drainage of the eye fluid. What are known as sulcus IOLs exhibit an elevated complication rate as a result of this and other effects.

By way of example, U.S. 2013/0226293 A1 describes an electroactive IOL, which is directly mechanically connectable to the ciliary muscle. Further, WO 2005/084587 A2 discloses a multi-part IOL comprising a plurality of optical elements which are able to be slid on one another for the purpose of changing the refractive power, the optical elements in each case being connected to the ciliary muscle of the eye via a support element.

### SUMMARY

It is an object of the present disclosure to provide an intraocular lens system which avoids the disadvantages afflicting the conventional IOLs.

According to the disclosure, this object is achieved by an intraocular lens system, a ciliary body implant, an intraocular lens, and a method for implanting an intraocular lens system into an eye, wherein a refractive power of the intraocular lens can be controlled via an interaction between a ciliary magnet element and a magnetic lens element in the eye.

In a first aspect, the disclosure relates to an intraocular lens system for implantation into an eye. The intraocular lens system comprises a ciliary body implant having a ciliary magnet element, the ciliary body implant being implantable into the eye in such a way that the ciliary magnet element at least partly follows a movement of the ciliary muscle or ciliary body. Moreover, the intraocular lens system comprises an intraocular lens comprising a magnetic lens element. In this case, the ciliary body implant and the intraocular lens are formed separately from one another and the intraocular lens system is configured to control a refractive power of the intraocular lens via an interaction between the ciliary magnet element and the magnetic lens element in the eye.

In a further aspect, the disclosure relates to a ciliary body implant for an intraocular lens system, the ciliary body implant comprising a ciliary magnet element and being configured to control a refractive power of the intraocular lens via an interaction between the ciliary magnet element and a magnetic lens element of an intraocular lens of the intraocular lens system.

In a further aspect, the disclosure relates to an intraocular lens for an intraocular lens system, the intraocular lens comprising a magnetic lens element and being configured to control a refractive power of the intraocular lens via an interaction between the magnetic lens element and a ciliary magnet element of a ciliary body implant of the intraocular lens system.

In a further aspect, the disclosure relates to a method for implanting an intraocular lens system into an eye. The method comprises an implantation of an intraocular lens into the eye, the intraocular lens comprising a magnetic lens element, and an implantation of a ciliary body implant with a ciliary magnet element into the eye, in such a way that the ciliary body implant at least partly follow a movement of the ciliary muscle or ciliary body. In this case, the ciliary body implant and the intraocular lens are formed separately from one another and the intraocular lens system is configured to control a refractive power of the intraocular lens via an interaction between the ciliary magnet element and the magnetic lens element in the eye.

Within the meaning of the disclosure, an intraocular lens system is a system comprising a biomechanically accommodatable intraocular lens (IOL) and one or more further elements for detecting the desire to accommodate, such as a ciliary body implant in particular, and for implementing the accommodation of the IOL. In this case, the intraocular lens system (IOL system) according to the disclosure has a multi-part design, with the plurality of parts of the IOL system being available as separate parts and, in particular, being implantable into the eye separately from one another. Typically, the plurality of parts of the IOL system, in particular the IOL and the ciliary body implant, require no direct mechanical and/or hydraulic and/or “wired” electrical connection to one another.

In this case, the ciliary body implant is an implant that is implantable into the eye and that at least partly follows the movement of the ciliary body. In this case, it is not mandatory for the ciliary body implant to be implanted and/or arranged directly in and/or on the ciliary body. Rather, indirect mechanical contact between the ciliary body implant and the ciliary body of the eye may also be sufficient, for as long as the ciliary body implant at the implanted site at least partly follows the movements of the ciliary body. In this case, the ciliary body implant typically fulfills the function of generating a signal from the movements of the ciliary body, the signal indicating the desire to accommodate and being able to be used for the accommodation of the IOL or of the eye.

In this case, the ciliary magnet element is a magnetic element which is integrated in an/or connected to the ciliary body implant and which generates a magnetic field. This magnetic field can then typically be used to provide the signal for indicating the desire to accommodate, by virtue of the ciliary magnet element at least partly following the movements of the ciliary body. In this case, the ciliary magnet element at least partly following the movements of the ciliary body means that a deflection of the ciliary magnet element or the change in position thereof in the eye need not necessarily be with the same amplitude and/or in the same direction as the deflection of the ciliary body which causes the deflection and/or the change in position of the ciliary body. Rather, it may be sufficient for the ciliary magnet element to follow the movements of the ciliary body in such a way that the ciliary magnet element provides a signal which allows at least qualitative identification of the movement of the ciliary body. Typically, the signal provided by the ciliary magnet element, for instance a change in the magnetic field generated by the ciliary magnet element, is proportional, typically directly proportional, to the amplitude of the causal movement of the ciliary body.

Typically, the ciliary magnet element is designed as a permanent magnet or comprises the latter. By way of example, the permanent magnet may comprise a ferromagnetic material, for instance a ferrite.

In this case, the IOL is an accommodatable IOL, particularly typically a biomechanical

IOL. Thus, the eye can accommodate, in particular by way of a change in the refractive power of the IOL in the eye. In this case, the change in the refractive power can typically be implemented by virtue of a mechanical force being exerted on the IOL or at least a part of the IOL. In this case, the mechanical force can be provided by the magnetic field generated by the ciliary magnet element, or by a change in the magnetic field, for example.

Typically, the magnetic lens element is designed as a permanent magnet or comprises the latter. By way of example, the permanent magnet may comprise a ferromagnetic material, for instance a ferrite. Particularly typically, the magnetic lens element and the ciliary magnet element are designed in the same way and, once implanted into an eye, are arranged in such a way that the respective magnetic field poles are arranged opposite to one another, that is to say in opposite direction to one another.

The interaction between the ciliary magnet element and the magnetic lens element typically comprises a magnetic interaction or consists of same. Optionally, one or more further interactions between the ciliary magnet element and the magnetic lens element may also be present, for instance an electrical interaction, in particular a capacitive interaction.

Thus, the interaction between the ciliary magnetic element and the magnetic lens element typically leads to, in particular, a change in the magnetic field generated by the ciliary magnet element, the change being caused by a positional change of the ciliary magnet element (at the position of the magnetic lens element) brings about an application of force on the magnetic lens element which then can be used to change the refractive power of the IOL and typically can be used for a corresponding accommodation of the eye, or the application of force brings this about. In this case, the interaction between the ciliary magnet element and the magnetic lens element controlling the refractive power of the IOL means that, typically, a change in the refractive power of the IOL follows a change in the interaction between the ciliary magnet element and the magnetic lens element, in particular a change in the magnetic interaction between the ciliary magnet element and the magnetic lens element. A movement of the ciliary body can typically be used in this way to control the refractive power of the IOL and particularly typically be used to provide the force for changing the refractive power of the IOL.

The disclosure offers the advantage that the IOL system can be provided with passive components only. In particular, the interaction between the ciliary magnet element and the magnetic lens element can be implemented by means of permanent magnets such that the IOL system requires no active component. This offers the advantage that there is no need to provide a power storage unit, for instance a rechargeable battery and/or a battery, and, accordingly, there also is no need to replace or exchange such a power storage unit. This can simplify the provision of the IOL system and/or reduce the maintenance outlay required.

The disclosure also offers the advantage that the IOL system can be implanted into an eye in such a way that there is no need for direct contact between the IOL system and the iris and/or no need for relative movement between the IOL system, in particular the ciliary body implant, and critical tissue in the eye. Complications can thus be avoided since the IOL system does not detach pigments from the iris and consequently there is no obstacle for the drainage of the eye fluid through the IOL system. As a result, the IOL system according to the disclosure allows for a reduction in the complication rate in comparison with conventional accommodatable IOLs.

Moreover, the disclosure offers the advantage that the accommodatable IOL can be designed in a compact form. In an exemplary embodiment, an implantation of the IOL into the capsular bag is facilitated, thereby promoting a low complication rate. This is moreover promoted by the fact that an IOL system according to the disclosure requires no direct mechanical and/or electrical connection between the ciliary body implant and the IOL, and accordingly there is no need to run a mechanical connection or electrical conductors from the IOL to the ciliary body implant through the capsular bag. This is advantageous since damage to the capsular bag and possible complications accompanying this can be avoided or reduced. Moreover, this offers the advantage that it is sufficient to merely implant the IOL into the capsular bag and there is no need or cause to implant the ciliary body implant into the capsular bag. In this way, an incision in the capsular bag required for the implantation of the IOL into the capsular bag can be kept small.

Furthermore, the disclosure offers the advantage that a transfer of force from the ciliary body to the IOL for the purposes of changing the refractive power of the IOL and hence for the accommodation of the eye need not necessarily be implemented via the zonular fibers and/or the capsular bag of the eye. Rather, the disclosure facilitates a transfer of force by way of the interaction, in particular magnetic interaction, between the ciliary magnet element and the magnetic lens element, the transfer not being significantly influenced by (unknown) individual tissue properties of the zonular fibers and/or of the capsular bag. As a result, falsifications and/or individual force differences of the force transfer from the ciliary body to the IOL can be reduced or even avoided.

Typically, the refractive power of the intraocular lens is controlled by virtue of the interaction between the ciliary magnet element and the magnetic lens element moving two or more Alvarez plates relative to one another in the intraocular lens. To this end, two or more Alvarez plates can be integrated into the intraocular lens or the intraocular lens can consist of two or more Alvarez plates. The Alvarez plates may have a rigid design such that these maintain their form unchanged in the case of a displacement relative to one another. Alternatively, the Alvarez plates can have an at least partly elastic form such that these partly change their shape when the Alvarez plates are displaced relative to one another. The Alvarez plates offer the advantage that these may provide a simple and cost-efficient option for controlling the refractive power in an intraocular lens.

Optionally, an intraocular lens can be designed to have a (static) cylindrical power in addition or as an alternative to a spherical power. According to an exemplary embodiment, the cylindrical or toric power of the intraocular lens can be adjustable by way of relative positioning of the Alvarez plates in a direction perpendicular to the movement direction or displacement direction of the Alvarez plates. By way of example, the intraocular lens can be designed in such a way that the cylindrical power can be set by an offset arrangement of the Alvarez plates relative to one another. In this case, the relative offset of the Alvarez plates to one another is implemented in a direction perpendicular to the displacement direction in which the Alvarez plates are moved relative to one another for controlling the (spherical) refractive power, and perpendicular to the optical axis of the intraocular lens. Since cylindrical accommodation is typically not desired for intraocular lenses, the offset of the Alvarez plates for setting the cylindrical power is optionally set prior to the insertion of the intraocular lens and then remains unchanged following the insertion or implantation of the intraocular lens. Expressed differently, the Alvarez plates are optionally not movable relative to one another along the offset for setting the cylindrical power. Setting the cylindrical power of the Alvarez lenses in this way by means of the relative offset offers the possibility of providing an optional cylindrical power using standardized Alvarez plates.

According to other exemplary embodiments, the cylindrical refractive power of the intraocular lens can be provided in another way, that is to say independently of a possible offset of the Alvarez plates relative to one another. By way of example, the intraocular lens may to this end be formed with a further optical element that provides the cylindrical power. In this case, this further optical element can be formed separately from the Alvarez plates and may likewise be integrated into the intraocular lens. Alternatively or in addition, the further optical element may exist in a corresponding exemplary embodiment of the Alvarez plates which provides the offset-independent cylindrical power. By way of example, this may be implemented by appropriate shaping of the Alvarez plates such that these have a cylindrical refractive power independently of the offset and/or by way of suitable diffractive patterning on one or more Alvarez plates, which then provide a static, cylindrical, diffractive power in addition to the variable, spherical power of the Alvarez plates. Here, the cylindrical power being static means that the cylindrical power remains unchanged when the eye accommodates. According to further exemplary embodiments, the provision of the static cylindrical power can also be provided by a combination of an offset-dependent cylindrical, refractive power of the Alvarez plates and a further optical element, for instance diffractive patterning.

Optionally, an alignment of the cylinder axis of a cylindrical power of the intraocular lens is definable by a fixed orientation of the intraocular lens relative to the eye. In particular, if Alvarez plates are used to control the refractive power, the intraocular lens can be implanted in such a way that the axis position of the cylindrical power extends in the desired direction. The relative angular position of the displacement direction in which the Alvarez lenses are displaced for the purposes of controlling the refractive power has no influence on the optical power of the intraocular lens and may therefore remain unconsidered when choosing the relative angular position of the intraocular lens during the implanting process.

Typically, the refractive power of the intraocular lens is controlled by virtue of the interaction between the ciliary magnet element and the magnetic lens element changing a shape of a membrane in the intraocular lens. This exemplary embodiment can be advantageous for fluid-filled lenses in particular, in which the geometric arrangement of the fluid, and hence the lens shape, can be changed by means of the membrane.

Alternatively or in addition, the refractive power of the intraocular lens can typically be controlled by virtue of the interaction between the ciliary magnet element and the magnetic lens element changing a spacing of two optical components of an optical doublet in the intraocular lens. Alternatively or in addition, the refractive power of the intraocular lens can typically be controlled by virtue of the interaction between the ciliary magnet element and the magnetic lens element changing the shape of the intraocular lens, which may be advantageous for thin and/or flexible lenses in particular. However, in addition to these exemplary embodiments explicitly mentioned, other mechanisms which facilitate a reliable change in the refractive power of the lens with little force outlay are also usable. According to further exemplary embodiments, one or more of these options for controlling the refractive power can be combined with the optional use of Alvarez plates. By way of example, the Alvarez plates may be embedded in a fluid-filled membrane such that, when the eye accommodates, firstly the Alvarez plates are displaced relative to one another and secondly the shape of the fluid-filled membrane changes, and both effects together bring about the desired change in the refractive power of the intraocular lens.

Typically, the ciliary body implant is implantable into the eye in such a way that the ciliary magnet element is in mechanical contact with the ciliary body and/or with the sulcus. By way of example, the ciliary body implant can be fastened directly in the ciliary body and/or can be positioned in the sulcus and/or in the vicinity of the sulcus. This offers the advantage that an avoidance of mechanical contact between the ciliary body implant and the iris can be attained particularly reliably. This also offers the advantage that the ciliary magnet element can follow the movements of the ciliary body particularly reliably without, for example, there being a falsification by the zonular fibers and/or the capsular bag. The method for implanting the IOL system is typically implemented in such a way here that the ciliary magnet element is in mechanical contact with the ciliary body and/or with the sulcus.

Typically, the ciliary body implant comprises a plurality of ciliary magnet elements which are arrangeable so as to be spaced apart from one another in mechanical contact with the ciliary body and/or with the sulcus. This offers the advantage that an adjacent arrangement of the magnetic lens element with one of the ciliary magnet elements can be simplified since accurate positioning of an individual, specific ciliary magnet element adjacent to the magnetic lens element and, accordingly, an accurate positioning in the circumferential direction of the IOL are not mandatory. As a result, the precision required when implanting the ciliary body implant and/or the implantation complexity can be reduced. Typically, the implantation is implemented in such a way that the magnetic lens element and the ciliary magnet element are arranged adjacently in a direction perpendicular to the optical axis of the intraocular lens.

Typically, the plurality of ciliary magnet elements are elastically interconnected and arranged in the ciliary body implant in ring-shaped or circular segment-shaped fashion and/or opposite one another relative to the optical axis of the intraocular lens. Such an arrangement and embodiment of the ciliary body implant offers the advantage that the latter can adapt in a particularly suitable manner to the contour of the ciliary body and, accordingly, the ciliary magnet elements can typically be arranged in direct mechanical contact with the ciliary body.

Typically, the ciliary body implant is designed in ring-shaped or circular segment-shaped fashion and a diameter and/or radius of curvature of the ciliary body implant is changeable by means of the elastic connections between the ciliary magnet elements and is typically adaptable to the ciliary body. By way of example, a ring-shaped ciliary body implant can be clamped into the ciliary body in the process and can abut against the inner surface of the latter, that is to say against the side facing the IOL, in circumferential fashion. Accordingly, a circular segment-shaped ciliary body implant can be arranged abutting against a part of the inner surface of the ciliary body, for example over 90°, 180°, or 270° of the inner circumference. In this case, a plurality of ciliary magnet elements can typically be arranged distributed over the entire circumference or the entire length of the ciliary body implant. In this case, the ciliary magnet elements can have the same or different designs. The ciliary magnet elements can typically be arranged equidistantly, that is to say with the same spacings from one another, in the ciliary body implant. Additionally, according to exemplary embodiments, a different element may be arranged in place of a ciliary magnet element at some positions, the different element not being a ciliary magnet element but, for example, a blind element which typically has the same spatial dimensions as a ciliary magnet element but has no function or a different function. Typically, the ciliary body implant has a ring-shaped or circular segment-shaped form and the implantation is implemented in such a way that the ciliary body implant is arranged in and/or on the ciliary body and/or in and/or on the sulcus.

Typically, the ciliary body implant and the intraocular lens are implantable into the eye in such a way that the at least one ciliary magnet element and the at least one magnetic lens element are arranged adjacently in a direction perpendicular to the optical axis of the intraocular lens and, typically, the magnetic dipoles of the ciliary magnet element and of the magnetic lens element are aligned opposite to one another. This offers the advantage of the interaction between the magnetic lens element and the adjacent ciliary magnet element being optimized. Typically, the magnetic lens element and the adjacent ciliary magnet element are arranged relative to one another in such a way that these exert a repulsive effect on one another.

Typically, the intraocular lens comprises a plurality of magnetic lens elements and a respective ciliary magnet element in the ciliary body implant is typically assigned to each magnetic lens element. By way of example, the IOL comprises two magnetic lens elements which are arranged at diametrically opposite positions of the IOL. This may offer the advantage of being able to obtain a particularly uniform force application and/or deformation and/or change in the refractive power of the IOL. Typically, the ciliary body implant and the IOL are arranged relative to one another in such a way that a ciliary magnet element is arranged adjacent to each magnetic lens element.

Typically, the intraocular lens comprises an optically transparent lens body and at least one extension, on and/or in which the at least one magnetic lens element is arranged. The extension may comprise a haptic or be formed as a haptic. By way of example, the at least one extension can extend radially to the outside from the lens body. By way of example, the lens extension can be formed lying in the same plane as the lens body. The extension offers the advantage that a magnetic lens element can be arranged in and/or on the IOL without the magnetic lens element covering part of the aperture of the IOL. The magnetic lens element is typically housed in the haptic. The haptic is typically embodied in such a way that the lens body can be well aligned and fixated in the capsular bag. Moreover, the haptic offers the option of arranging one or more magnetic lens elements in and/or on the haptic and accordingly also of fixating and positioning the magnetic lens elements in the eye together with the haptic.

The features and exemplary embodiments specified above and explained below should not only be considered to be disclosed in the respective explicitly mentioned combinations in this case, but are also comprised by the disclosure in other technically advantageous combinations and exemplary embodiments and on their own in each case.

### BRIEF DESCRIPTION OF THE DRAWINGS

The disclosure will now be described with reference to the drawings wherein:

FIG. 1 shows an exemplary embodiment of an intraocular lens system implanted into an eye, both in a longitudinal sectional view and in a transverse sectional view;

FIGS. 2A and 2B show an intraocular lens system according to an exemplary embodiment in various states of arrangement;

FIG. 3 shows an intraocular lens system according to an exemplary embodiment;

FIGS. 4A and 4B show an intraocular lens system according to an exemplary embodiment with Alvarez surfaces;

FIGS. 5A and 5B show an intraocular lens system according to an exemplary embodiment with a fluid-filled lens;

FIG. 6A to 6C show various exemplary embodiments of ciliary body implants;

FIG. 7 shows an intraocular lens according to an exemplary embodiment;

FIGS. 8A to 8C show schematic representations of Alvarez plates; and

FIG. 9 shows the relative position of an optional cylindrical power relative to the Alvarez plates.

### DESCRIPTION OF EXEMPLARY EMBODIMENTS

The same or similar elements in the various exemplary embodiments are denoted by the same reference signs in the drawings for reasons of simplicity.

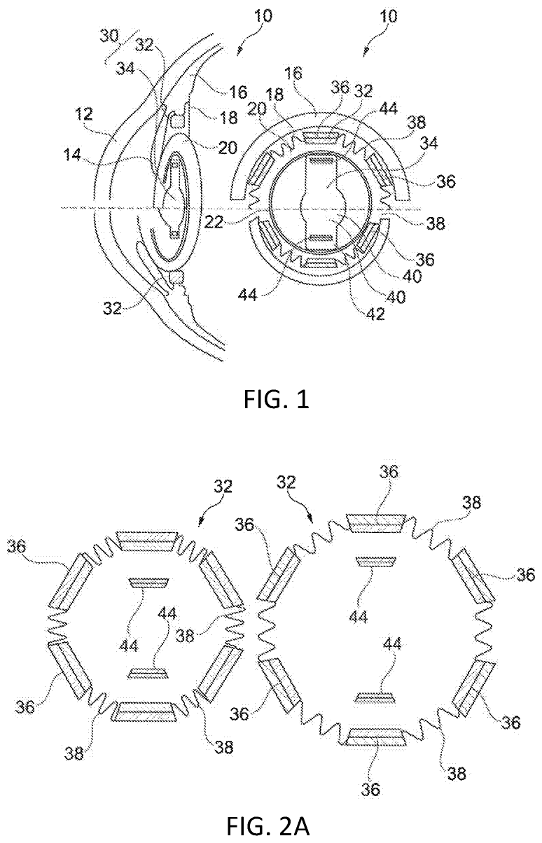

FIG. 1 shows a schematic representation of an eye 10 with an implanted intraocular lens system 30 (IOL system) according to an exemplary embodiment, in a longitudinal sectional view (left) along a sectional plane in which the optical axis 100 of the eye 10 runs, and in a transverse sectional plane (right) perpendicular to the optical axis 100.

The longitudinal sectional view of the eye 10 allows identification of the cornea 12 and the iris 14 of the eye 10, and the ciliary body 16 located therebehind, the zonular fibers 18 and the empty capsular bag 22, and the space where the crystalline lens 20 was arranged, the latter however having already been removed from the eye in the exemplary embodiment shown.

FIG. 1 is also divided in two in the vertical direction, the upper part of the longitudinal sectional view and of the cross-sectional view in each case showing the eye 10 in a first accommodated state and the lower part showing the eye 10 in a second accommodated state. By way of example, the state shown in the upper image half may represent a disaccommodated state of the eye. In exemplary fashion below, the first accommodated state should be considered to be a weakly accommodated state for distance accommodation, while the second accommodated state is considered to be a more strongly accommodated state for near accommodation.

Further, FIG. 1 shows the implanted intraocular lens system 30, which is of multi-part design and which comprises a ciliary body implant 32 and an intraocular lens (IOL) 34, the ciliary body implant 32 and the IOL 34 being formed separately from one another.

According to the exemplary embodiment shown, the ciliary body implant 32 comprises six ciliary magnet elements 36, which are elastically interconnected and arranged in such a way that the ciliary body implant 32 is designed as a ring-shaped structure. According to the exemplary embodiment shown, the ciliary magnet elements 36 are connected by means of mechanical spring elements 38. In this case, the elastic connection of the ciliary magnet elements 36 is designed in such a way that a compression and strain of the ciliary body implant 32 is rendered possible in the radial direction such that the ciliary body implant 32 can follow the movements of the ciliary body 16 when the eye 10 accommodates or transitions into a non-accommodated state.

In this case, the ciliary magnet elements 36 are arranged in such a way that all ciliary magnet elements 36 are poled in the same way in the radial direction. By way of example, all ciliary magnet elements can be arranged in such a way that their magnetic south poles point radially inward and their north poles point outward. According to other exemplary embodiments, the ciliary magnet elements 36 can also be arranged in such a way that their magnetic north poles point radially inward and the south poles point radially outward.

In this case, the ciliary body implant 32 is implanted with direct mechanical contact with the ciliary body 16 into the sulcus of the eye or on the sulcus of the eye outside of the capsular bag 22 such that a movement of the ciliary body 16 is transferred directly to the ciliary body implant 32 and the ciliary body implant accordingly follows the movements of the ciliary body 16 by way of a strain or compression. When following the movements of the ciliary body 16, the ciliary body implant 32 can be compressed or strained in such a way by the ciliary body 16 that the diameter of the ciliary body implant 32 increases or reduces, and so the ciliary body implant 32 rests against the inner side of the ciliary body 16 or against the sulcus.

The IOL 34 is arranged within the capsular bag 22 and comprises a lens body 40 as well as two extensions or haptics 42. A respective magnetic lens element 44 is arranged in the two extensions 42. According to other exemplary embodiments, the IOL 34 may also comprise only one or more than two extensions or haptics 42, in each of which one or more magnetic lens elements 44 are arranged.

In this case, the magnetic lens elements 44 and the ciliary magnet elements 36 are formed as permanent magnets or comprise one or more permanent magnets. The ciliary body implant 32 and the IOL 40 are arranged in such a way that each magnetic lens element 44 is arranged adjacently with a ciliary magnet element 36 in the radial direction in order to achieve the greatest possible interaction between the magnetic lens element 44 and the adjacent ciliary magnet element 36. In this case, it is typical if, like in the exemplary embodiment shown, the ciliary body implant 36 comprises a plurality of ciliary magnet elements 36, in particular more than two ciliary magnet elements 32, since this eases the arrangement of the ciliary body implant 32 and the IOL 34 relative to one another during the implantation, in such a way that a ciliary magnet element 36 is arranged adjacent to the respective magnetic lens elements 44 in each case, and hence this simplifies the implantation process. In this case, the magnetic fields of the respectively adjacent ciliary magnet elements 36 and magnetic lens elements 44 are aligned opposite to one another such that these repel.

In this case, the IOL system 30 facilitates a force transfer from the ciliary body 16 to the IOL 34 via the ciliary body implant 32. In particular, the force exerted by the ciliary body 16 on the ciliary body implant 32 in the process is transferred from the ciliary magnet elements 36 to the magnetic lens elements 44 of the IOL 34 by way of a magnetic interaction such that the force exerted by the ciliary body 16 acts on the magnetic lens elements 44 and this in turn changes the refractive power of the lens body 40 or of the IOL 34. Consequently, the implanted IOL system 30 offers the option of changing the refractive power of the IOL 34 by way of movements of the ciliary body 16, and of accommodating the eye in this way.

In the upper part of FIG. 1, the eye 10 is respectively shown in a weakly accommodated or disaccommodated state. In this case, the ciliary muscle 16 is relaxed and the IOL 34 is present in the state of distance refractive power or disaccommodation. The ciliary body implant 32 is likewise stretched or relaxed in the process and adjusts to the internal diameter of the ciliary body 16, and so the ciliary body implant 32 also has a large diameter (relative to the diameter in the accommodated state of the eye).

In the lower part of FIG. 1, the eye 10 is depicted in a second accommodated state in each case, with stronger accommodation being present in this state than in the upper half of the drawing. In this case, the ciliary muscle is more tensioned, as a result of which a radially inwardly directed force indirectly is exerted on the ciliary body implant 32 and, via the ciliary body implant 32, on the IOL 34. The interaction between the ciliary magnet elements 36 and the magnetic lens elements 44 in this case transfers the force at least in part to the IOL 34, as a result of which the IOL 34 is compressed in the radial direction and, as a result, the refractive power of the IOL 34 is increased so that the eye 10 accommodates more strongly.

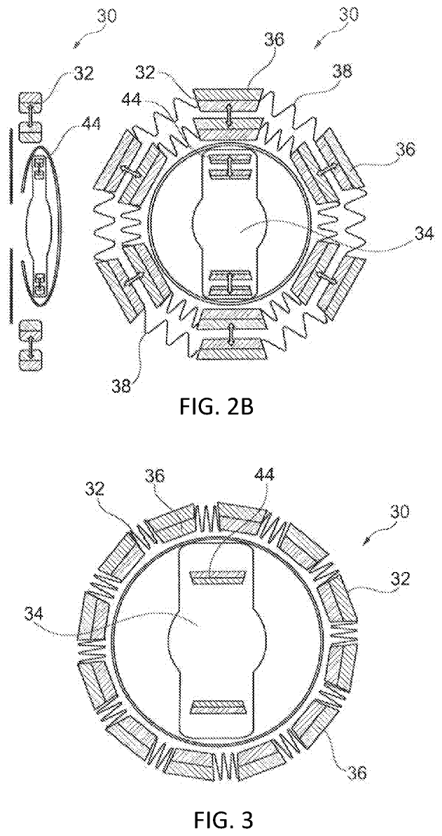

The movement of the ciliary body implant 32 and the resultant movement of the IOL 34 are explained on the basis of FIGS. 2A and 2B. FIG. 2A shows a schematic representation of a ciliary body implant 32 according to the exemplary embodiment shown in FIG. 1. On the left-hand side, the ciliary body implant 32 is shown in a radially compressed form, for example in an accommodated state. The right-hand side shows the ciliary body implant in a relaxed or stretched form, for example in a weakly accommodated state. FIGS. 2A and 2B likewise show the magnetic lens elements 44 which are located radially within the ciliary body implant 32 and which are repelled by the adjacent ciliary magnet elements 36 on account of magnetic interaction. Accordingly, the magnetic lens elements 44 also follow a radial movement of the ciliary magnet elements 36 and are pressed radially inward by the ciliary magnet elements 36 when the ciliary body implant 32 transitions into the compressed state, and are pushed radially outward again by a restoring force of the IOL 34 when the ciliary body implant 32 transitions back into the stretched state in the case of a relaxed eye 10. FIG. 2B elucidates the movement of the ciliary body implant 32 and the movement of the magnetic lens elements 44 caused thereby and the resultant application of force on the IOL 34 on the basis of an overlaid representation of the IOL system in the compressed state (inside) and in the relaxed state (outside).

FIG. 3 shows an IOL system 30 according to a further exemplary embodiment which has a ciliary body implant 32 having twelve ciliary magnet elements 36. Like in the previous exemplary embodiment, too, the ciliary magnet elements 36 are elastically interconnected by means of mechanical spring elements 38. On account of the greater number of twelve ciliary magnet elements 36 (in comparison with six ciliary magnet elements 36 in the previous exemplary embodiment), these have a shorter form in the circumferential direction since the maximum diameter and circumference of the ciliary body implant 32 is specified by the internal diameter of the ciliary body 16. In order to obtain the greatest possible magnetic interaction between the magnetic lens elements 44 and the respective adjacent ciliary magnet elements 36, accurate radially adjacent positioning of one of the ciliary magnet elements 36 with respect to the respective magnetic lens elements 44 is typical. When implanting the IOL system 30, such accurate positioning can for example be achieved by virtue of the fact that the ciliary body implant 36 is turned or rotated into the desired alignment around the center of the ring-shaped ciliary body implant 32 such that one of the ciliary magnet elements 36 is positioned adjacently to one of the magnetic lens elements 44 in the radial direction. The use of a large number of ciliary magnet elements 36 arranged along the circumferential direction moreover offers the advantage that the requirement of accurate relative positioning of a ciliary magnet element 36 with respect to a respective magnetic lens element 44 is reduced or dispensed with entirely since the magnetic field between the individual ciliary magnet elements 36 generated from the totality of the ciliary magnetic elements 36 is not attenuated, or only attenuated to a very small extent, in comparison with positions that directly adjoin a ciliary magnet element 36.

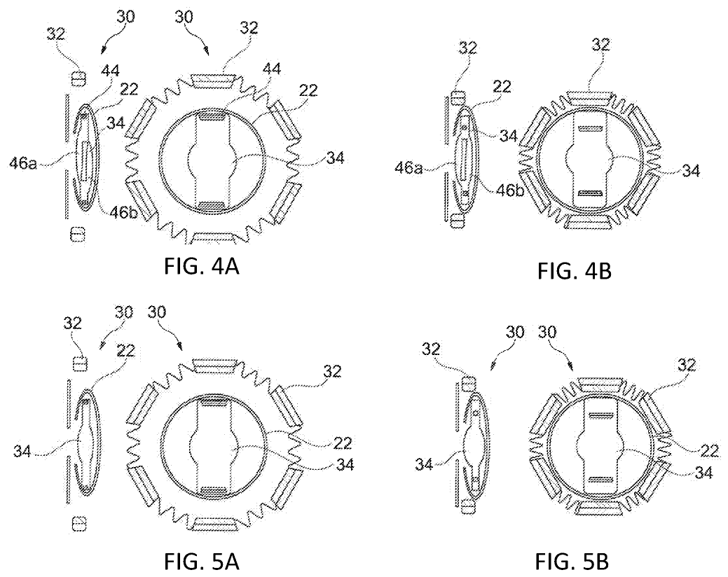

FIGS. 4A and 4B show an IOL system 30 according to an exemplary embodiment, in which the refractive power of the IOL is changeable by means of two displaceable Alvarez surfaces 46a and 46b. FIG. 4a shows, in a schematic longitudinal sectional view (left) and in a schematic cross-sectional view (right), the IOL system 30 in a relaxed state, for example when the eye 10 is relaxed and only weakly accommodated, for example in distance accommodation. FIG. 4B accordingly shows the IOL system 30 in a compressed state, for instance if the eye is accommodated for the near region.

In this case, the IOL 34 comprises two Alvarez surfaces 46a and 46b, each of which is connected to a magnetic lens element 44 by means of an extension comprising a haptic. If the ciliary body 16 does not exert a force on the IOL 34 by the ciliary body implant 32, the Alvarez surfaces 46a and 46b are pushed radially to the outside such that the IOL 34 has the lowest refractive power, for example for distance accommodation. By contrast, if the ciliary body 16 exerts a force on the IOL 34 via the ciliary body implant 32, the Alvarez surfaces 46a and 46b are pushed over one another in the radially inward direction, as a result of which there is an increase in the refractive power of the IOL 34 and near accommodation can be achieved.

FIGS. 5A and 5B shown an IOL system according to a further exemplary embodiment, in which the change in the refractive power is achieved by a change in the shape of the IOL 34. The IOL 34 is typically formed as an elastic, fluid-filled lens or comprises the latter. In particular, the IOL typically comprises a membrane, by means of which the shape of the fluid-filled lens is changeable. The membrane can be deformed by the ciliary body applying a force on the IOL 34 via the ciliary body implant 32, in such a way that the IOL is compressed in the radial direction and thereupon increases its refractive power. FIG. 5A shows the IOL system in a relaxed state in which the IOL has a low refractive power, while FIG. 5B shows the IOL system in a compressed state, in which the IOL 34 has a significant refractive power.

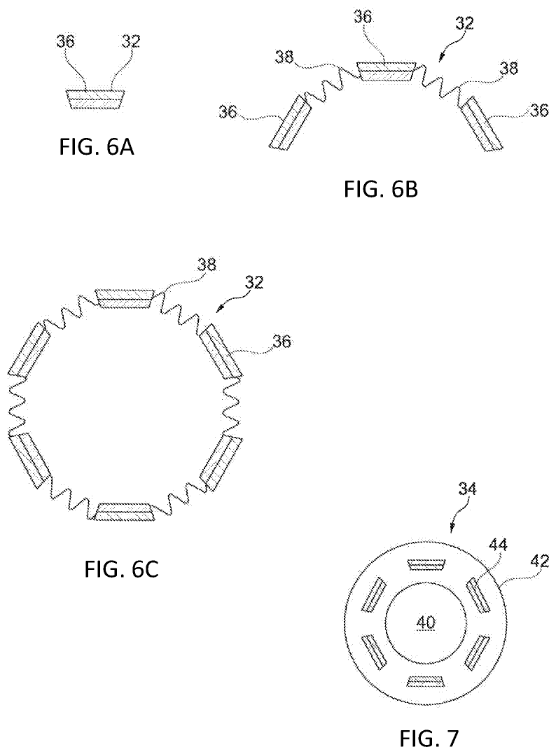

FIGS. 6A to 6C show exemplary embodiments of ciliary body implants 32 in exemplary fashion. FIG. 6A shows an exemplary embodiment in which the ciliary body implant comprises only one ciliary magnet element 36, which is attachable to the ciliary body, for example in and/or on the sulcus. FIG. 6B shows a circular segment-shaped ciliary body implant 32, which comprises three ciliary magnet elements 36 that are elastically connected by two mechanical spring elements 38. By way of example, the circular segment-shaped ciliary body implant can be arranged in and/or on the sulcus in such a way that the ciliary body implant 32 contacts a part of the ciliary body or its inner surface. FIG. 6C shows a further exemplary embodiment, according to which the ciliary body implant has a ring-shaped form and comprises six ciliary magnet elements 36 connected by way of elastic spring elements 38. By way of example, this ring-shaped ciliary body implant 32 can be arranged in and/or on the sulcus such that the ciliary body implant completely runs around the inner surface of the ciliary body 16.

FIG. 7 shows a further exemplary embodiment of an IOL 34 in a schematic representation. According to this exemplary embodiment, the IOL 34 comprises an inner lens body 40, which is surrounded along its entire perimeter by an extension 42 comprising a haptic. Six magnetic lens elements 44 are arranged in the circumferential direction within the extension 42. In particular, this exemplary embodiment facilitates a simplified alignment of the IOL 34 relative to the ciliary body implant since the alignment of the magnetic lens elements 44 in relation to the ciliary magnet elements 36 is simplified on account of the large number of magnetic lens elements 44.

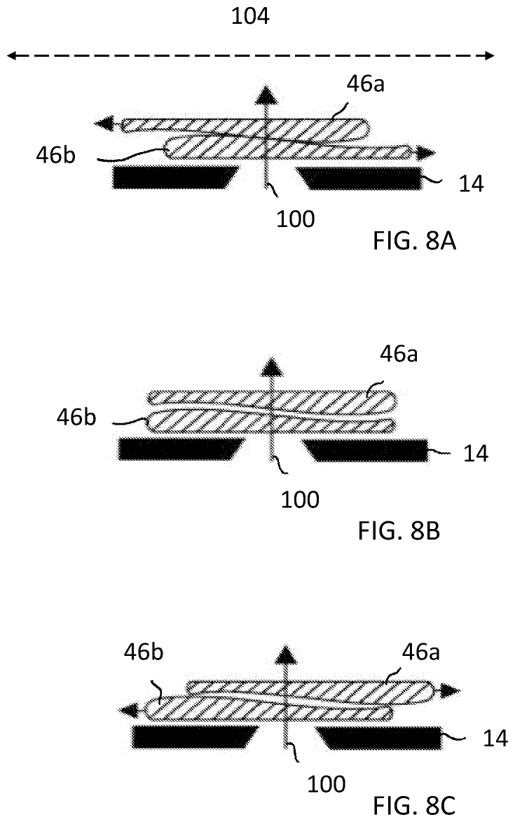

FIGS. 8A to 8C show the principle of action of an intraocular lens on the basis of Alvarez plates 46a and 46b in schematic representations. The relative positioning of the Alvarez plates 46a, 46b in the displacement direction 104 leads to a change in the refractive power of the Alvarez plate pair and consequently of the intraocular lens. In this case, increased overlap of the Alvarez plates 46a and 46b leads to an increased spherical, refractive power. By way of example, the intraocular lens can be set in such a way that the latter has positive refractive power in the position shown in FIG. 8A, no refractive power in the position shown in FIG. 8B and negative refractive power in the position shown in FIG. 8C. In this case, the Alvarez plates 46a, 46b are formed adjacent to the iris 14 and may optionally be integrated into the capsular bag and/or integrated into another, possibly fluid-filled membrane (not shown).

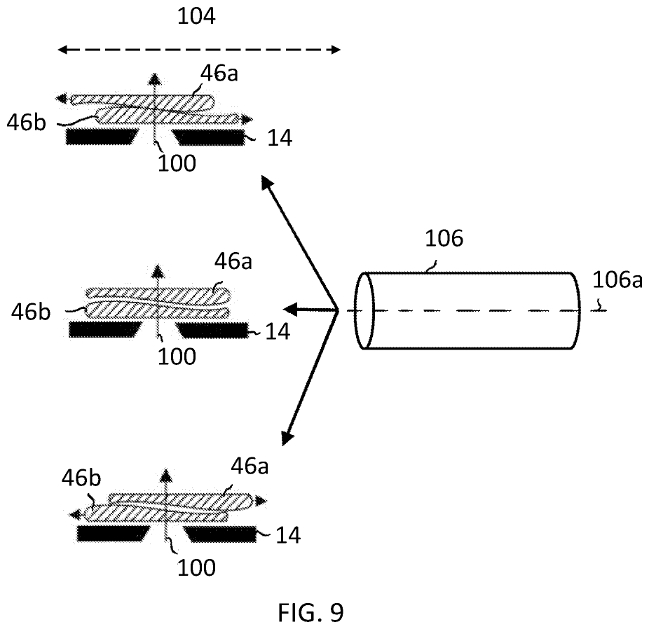

An optional, static, cylindrical power can for example be provided by an optional further optical element (not shown) of the intraocular lens 34 if desired. FIG. 9 shows the relative position of the cylindrical power, symbolized by a cylinder 106.

In this case, it is possible to identify that the cylinder axis 106a extends parallel to the displacement direction 104 of the Alvarez plates 46a, 46b. What this achieves is that the cylindrical power remains unchanged when the Alvarez plates 46a, 46b are displaced for the purposes of changing the spherical power.

According to a further exemplary embodiment, the cylindrical power can be formed by an offset of the Alvarez plates 46a, 46b relative to one another perpendicular to the displacement direction 104 and perpendicular to the optical axis 100, that is to say out of the plane of the drawing or into the plane of the drawing, as an alternative or in addition to a further optical element. In the process, this may lead to a different manifestation of the cylindrical power of the intraocular lens, and so the cylindrical power can be set by way of the offset of the Alvarez plates before the intraocular lens is inserted.

The foregoing description of the exemplary embodiments of the disclosure illustrates and describes the present invention. Additionally, the disclosure shows and describes only the exemplary embodiments but, as mentioned above, it is to be understood that the disclosure is capable of use in various other combinations, modifications, and environments and is capable of changes or modifications within the scope of the concept as expressed herein, commensurate with the above teachings and/or the skill or knowledge of the relevant art.

The term “comprising” (and its grammatical variations) as used herein is used in the inclusive sense of “having” or “including” and not in the exclusive sense of “consisting only of.” The terms “a” and “the” as used herein are understood to encompass the plural as well as the singular.

All publications, patents and patent applications cited in this specification are herein incorporated by reference, and for any and all purposes, as if each individual publication, patent or patent application were specifically and individually indicated to be incorporated by reference. In the case of inconsistencies, the present disclosure will prevail.

### LIST OF REFERENCE SIGNS

10 Eye
12 Cornea
14 Iris
16 Ciliary body
18 Zonular fibers
20 Crystalline lens
22 Capsular bag
30 Intraocular lens system (IOL system)
32 Ciliary body implant
34 Intraocular lens (IOL)
36 Ciliary magnet element
38 Mechanical spring element
40 Lens body
42 Extension
44 Magnetic lens element
46a, 46b Alvarez surface or Alvarez plate
100 Optical axis of the eye
102 Rotational direction for aligning the IOL system
104 Displacement direction of the Alvarez plates
106 Cylinder for representing the cylindrical power
106a Cylinder axis

1. An intraocular lens system for implantation in an eye, the intraocular lens system comprising:
a ciliary body implant having a ciliary magnet element, the ciliary body implant being implantable into the eye such that the ciliary magnet element at least partly follows a movement of the ciliary body of the eye; and
an intraocular lens having a magnetic lens element, the ciliary body implant and the intraocular lens being formed separately from one another and the intraocular lens system being configured to control a refractive power of the intraocular lens via an interaction between the ciliary magnet element and the magnetic lens element in the eye.

2. The intraocular lens system as claimed in claim 1, wherein the refractive power of the intraocular lens is controlled via an interaction between the ciliary magnet element and the magnetic lens element moving two or more Alvarez plates in the intraocular lens relative to one another.

3. The intraocular lens system as claimed in claim 2, wherein a cylindrical power of the intraocular lens is adjustable by way of relative positioning of the Alvarez plates in a direction perpendicular to the movement direction of the Alvarez plates and/or is provided by a further optical element of the intraocular lens.

4. The intraocular lens system as claimed in claim 1, wherein an alignment of a cylinder axis of the cylindrical power of the intraocular lens is definable by a fixed orientation of the intraocular lens relative to the eye.

5. The intraocular lens system as claimed in claim 1, wherein the refractive power of the intraocular lens is controlled by:
changing a shape of a membrane in the intraocular lens; and/or
changing a distance between two optical components of an optical doublet in the intraocular lens; and/or
changing the shape of the intraocular lens.

6. The intraocular lens system as claimed in claim 1, wherein the ciliary body implant is implantable into the eye such that the ciliary magnet element is in mechanical contact with a ciliary body and/or with a sulcus.

7. The intraocular lens system as claimed in claim 1, wherein the intraocular lens is implantable into the capsular bag of the eye.

8. The intraocular lens system as claimed in claim 1, wherein the ciliary body implant comprises a plurality of ciliary magnet elements which are arranged spaced apart from one another and in mechanical contact with the ciliary body and/or with the sulcus.

9. The intraocular lens system as claimed in claim 8, wherein the plurality of ciliary magnet elements is elastically interconnected and is arranged in the ciliary body implant in ring-shaped or circular segment-shaped fashion and/or opposite one another relative to the optical axis of the intraocular lens.

10. The intraocular lens system as claimed in claim 9, wherein the ciliary body implant is configured in a ring-shape or in circular segments and a diameter and/or a radius of curvature of the ciliary body implant is configured to be changeable by elastic connections between the ciliary magnet elements.

11. The intraocular lens system as claimed in claim 1, wherein the ciliary body implant is implantable into the eye in such a way that there is no direct mechanical contact between the ciliary body implant and the iris of the eye.

12. The intraocular lens system as claimed in claim 1, wherein the ciliary body implant and the intraocular lens are implantable into the eye such that the at least one ciliary magnet element and the at least one magnetic lens element are arranged adjacently in a direction perpendicular to the optical axis of the intraocular lens.

13. The intraocular lens system as claimed in claim 1, wherein the intraocular lens comprises a plurality of magnetic lens elements.

14. The intraocular lens system as claimed in claim 1, wherein the intraocular lens comprises an optically transparent lens body and at least one extension comprising a haptic, the at least one magnetic lens element being arranged on and/or in the extension.

15. A ciliary body implant for an intraocular lens system, the ciliary body implant comprising a ciliary magnet element and being designed to control a refractive power of the intraocular lens by means of an interaction between the ciliary magnet element and a magnetic lens element of an intraocular lens of the intraocular lens system.

16. An intraocular lens for an intraocular lens system, the intraocular lens comprising a magnetic lens element and being designed to control a refractive power of the intraocular lens by means of an interaction between the magnetic lens element and a ciliary magnet element of a ciliary body implant of the intraocular lens system.

17. The intraocular lens system as claimed in claim 10, wherein the ciliary body implant is configured to be adaptable to match the ciliary body.

18. The intraocular lens system as claimed in claim 12, wherein the magnetic dipoles of the ciliary magnet element and of the magnetic lens element are aligned opposite to one another.

19. The intraocular lens system as claimed in claim 13, wherein a respective ciliary magnet element in the ciliary body implant is assigned to each magnetic lens element.

20. A method for implanting an intraocular lens system into an eye, the method comprising:
implanting an intraocular lens into the eye, the intraocular lens comprising a magnetic lens element,
implanting a ciliary body implant with a ciliary magnet element into the eye, in such a way that the ciliary body implant at least partly follows a movement of the ciliary body,
the ciliary body implant and the intraocular lens being formed separately from one another and the intraocular lens system being configured to control a refractive power of the intraocular lens via an interaction between the ciliary magnet element and the magnetic lens element in the eye.
